(19) United States
(12) Patent Application Publication
(10) Pub. No.: US 2025/0367244 A1
(43) Pub. Date: Dec. 4, 2025

(54) COMBINED USE OF A PROBIOTIC AND BUTYRIC ACID TO INCREASE THE ENTEROCYTE'S HEALTH AND CONSEQUENTLY THE WELLNESS OF PETS AND LIVESTOCK

(71) Applicant: CERBIOS-PHARMA SA, Barbengo-Lugano (CH)

(72) Inventors: Raffaella COPPOLECCHIA, Barbengo-Lugano (CH); Claudio ISENBURG, Barbengo-Lugano (CH); Luca ANTONIOLI, Pisa (PI) (IT); Matteo FORNAI, Pisa (PI) (IT); Carolina PELLEGRINI, Pisa (PI) (IT)

(21) Appl. No.: 18/874,238

(22) PCT Filed: Jun. 20, 2023
(86) PCT No.: PCT/EP2023/066567

§ 371 Date: Dec. 12, 2024

(30) Foreign Application Priority Data
Jun. 22, 2022 (IT) 102022000013144

(51) Int. Cl. A61K 35/744; A61K 31/19; A61K 35/00; A61P 3/00; C12N 1/20; C12R 1/46

(52) U.S. Cl. CPC A61K 35/744; A61K 31/19; A61P 3/00; C12N 1/205; A61K 2035/115; C12R 2001/46

(57) ABSTRACT

The use of a probiotic, particularly the Enterococcus faecium strain NCIMB 10415 which is named SF68® as probiotic active ingredient, in combination with butyric acid or a salt thereof to increase the enterocyte's health and consequently the wellness of pets and livestock is described.

The present invention relates to animal nutritional field and more particularly to the use of a probiotic in combination with butyric acid or a salt thereof to increase the enterocyte's health and consequently the wellness of pets and livestock.

Butyric acid and salts thereof, particularly sodium butyrate, are used since decades for animal feeding. For an optimal use the animal needs to adsorb butyric acid or its salts through an uptake mechanism of this short chain fatty acid (SCFA) into the enterocytes.

However, the uptake of butyric acid or salts thereof at intestinal level can be impaired by inflammatory conditions, which cause or are characterized by damage of the intestinal tissue and epithelial barrier.

For example, we observed that the uptake of butyric acid or its salts is impaired in mice which are fed with high-fat diet and develop obesity.

Therefore, there is the need to optimize the use of butyric acid and its salts in animal feeding, particularly for pets (dogs, cats) and livestock (fattening turkeys, chickens, lying hens, sows and piglets (suckling and weaned), fattening pigs, milk cows, calves, etc.) to increase the enterocyte's health.

We have now found that the uptake of butyric acid or salts thereof at intestinal level in livestock and pets can be improved by simultaneously administering to the animal a probiotic, particularly an Enterococcus faecium strain, more particularly the Enterococcus faecium strain NCIMB 10415 which is named SF68® as probiotic active ingredient.

Therefore, object of the present invention is the use of Enterococcus faecium in combination with butyric acid or its salts as feed supplements to increase the enterocyte's health and consequently the wellness of pets and livestock.

In the present context, “to increase the enterocyte's health” means to increase the enterocyte's ability to uptake butyric acid. This results in a better wellness of the animals which, from a practical point of view, can mean a weight increase in livestock as well as an improvement of the physiological and behavioral parameters known to be a wellness index in pets such as, for example, appetite, sleep, skin/fur condition, etc. These wellness indexes in animals are well known to the expert in the field.

Preferably the Enterococcus faecium strain with access number NCIMB 10415, which is named SF68 as probiotic active ingredient, is used. Still more preferably, SF68 is used in microencapsulated form, for example, as that on the market under the name Cervinet® LBC ME10, LBC ME20 plus and LBC ME5 PET.

Butyric acid is preferably used in the form of salt, particularly sodium salt. Its use in incapsulated form is particularly preferred, for example in the formulation on the market under the name ButiPEARL®.

In the preferred embodiment of the present invention both the probiotic and butyric acid or its salts are given to the animal mixed with the standard diet.

The amount of probiotic and butyric acid or its salts will change depending from the kind of animal and the applicable local regulations.

For informational purposes only, without any limitation, we report in the following Table 1 some examples of doses of butyrate (ButiPEARL®) and SF68 (LBC ME10 and LBC ME20 plus for livestock and LBC ME5 PET for pets).

TABLE 1

| | ButiPEARL | E. faecium dose (ppm) | |
|---|---|---|---|
| Livestock | dose (ppm) | LBC ME10 | LBC ME20 plus |
| Broilers | 150-300 | 30-60 | 15-30 |
| Turkeys | 150-300 | 30-60 | 15-30 |
| Lying hens | 100-200 | 15-30 | 7.5-15 |
| Sows | 200-400 | 70-140 | 35-70 |
| Suckling piglets | 100-200 | 100-200 | 50-100 |
| Weaned piglets | 150-300 | 35-70 | 17.5-35 |
| Fattening pigs | 200-400 | 35-70 | 17.5-35 |
| Calves | 150-300 | 100-200 | 50-100 |
| Milk cows | 200-400 | 150-300 | 75-150 |

| | ButiPEARL | E. faecium dose (ppm) |
|---|---|---|
| Pets | dose (ppm) | LBC ME5 PET |
| Dogs | 150-300 | 500-1000 |
| Cats | 150-300 | 1400-2800 |

In its preferred embodiment, the present invention consists in feeding the animal with a standard feed, selected by the expert in the field on the basis of the animal type and its breeding characteristics (dairy, meat, etc.), supplemented with butyric acid or its salts and the probiotic SF68 in the abovementioned amounts, selected on the basis of the animal type as well.

A physiological growth and/or weight increase of the animal, which is improved compared to the animals fed with standard feed only, is obtained.

The idea of combining probiotic SF68 and butyric acid or salts thereof derives from experimental studies on mice treated with high-fat diet, wherein it has been found that butyric acid is better used/up-taken by the enterocytes when administered together with SF68 (see Example 1).

Based on the results on mice, we could observe that SF68 leads to a normalization of the expression of the apical transporter of butyrate, altered by the high-fat diet (HFD).

In further studies on livestock, in particular chickens, it has been observed that the use of SF68 together with butyric acid or salts thereof allows to obtain a weight increase of the animals compared to the controls receiving standard feed only.

This further feature of the supplementation of the animal feeding with butyric acid or salts thereof and probiotic Enterococcus faecium (SF68) makes the present invention particularly advantageous.

The utility, efficacy and advantages of the present invention will be now illustrated in greater details by the following examples which however are not meant to limit in any way the scope of the present invention.

### EXAMPLE 1

SF60 Reduces the Fecal Levels of Butyrate in Mice Fed with High-Fat Diet (HFD)

C57BL/6 mice (20-22 g weight), 5-week old, were provided by Envigo srl (San Pietro al Natisone, Udine, Italy). The mice were housed six in a cage in a temperature-controlled room on a 12-hour light cycle at 22-24° C., and 50-60% humidity and let familiarize for at least one week. The animals were handled following the Directive 2010/63/UE.

Standard diet (SD, 18% calories from fat; TD.2018) was administered during the adaptation period to all mice. Then, the animals were randomly divided into six groups, each composed by 10 mice, as follows:
SD for 8 weeks
HFD for 8 weeks
SD+SF68 (P) starting from the fourth week (treatment 4+4)
HFD+SF68 (P) starting from the fourth week (treatment 4+4)
SD+SF68 (P) for 8 weeks
HFD+SF68 (P) for 8 weeks

The high-fat diet (HFD) provided 60% calories from fat (TD.06414).

The comparison between the calorie counts of the two diets is reported herein below:

| | Kcal from | | |
|---|---|---|---|
| Diet | carbohydrates | Kcal from proteins | Kcal from fats |
| SD | 58% | 24% | 18% (3.1 kcal/g) |
| HFD | 21.4% | 18.3% | 60.8% (5.1 kcal/g) |

The body weights of the animals were measured once a week from the first day of the study. At the end of the 8 weeks, the animals were anaesthetized and sacrificed. Blood samples and tissue samples were collected and stored at −80° C. for further analysis.

For the analysis of short chain fatty acids (SCFA), fecal samples were lyophilized, and gas chromatography was performed. The lyophilizate were solubilized in 100 ml 5 M formic acid and 400 ml acetone and centrifuged (5 min at 4000 rpm). Using a GC2010 Plus gas chromatograph (Shimadzu Deutschland GmbH, Duisburg, Germany) equipped with a flame ionization detector with a thin-film capillary column Stabilwax® (Restek, Bad Homburg, Germany), the concentration of SCFA in the supernatants was determined. The samples were spread out by split injection using the auto-sampler AOC-20s/l (Shimadzu Deutschland GmbH). GC solution Chromatography Data System (Shimadzu Deutschland GmbH) was used for data processing. For the quantification of SCFA an external standard (Supelco™ WSFA-1 Mix, Supelco Sigma-Aldrich Co., Bellefonte PA) was employed.

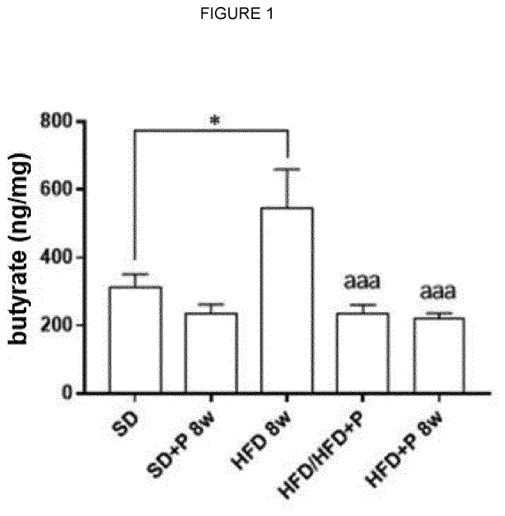

The results are reported in FIG. 1.

The mice fed with high-fat diet (HFD) showed a significant increase in the fecal butyrate levels after 8 weeks of feeding with HFD. Such an increase resulted to be antagonized by the supplementation with SF68 both in the treatment 4+4 and in the 8 week treatment.

### EXAMPLE 2

### Efficacy Study on Weight Increase in Chickens

Pen numbers for 5 treatment groups of chickens were randomly assigned to 12 replicates for a total of 60 study pens.

TABLE 2
Study design

| | Butyrate | E. faecium | | | | | | | | | | | | | |
|---|---|---|---|---|---|---|---|---|---|---|---|---|---|---|---|
| Group | (ppm) | (ppm) | Code | (Replicates: 12) | | | | | | | | | | | |
| 1 | 0 | 0 | WINE | 37 | 40 | 42 | 43 | 45 | 48 | 50 | 51 | 54 | 55 | 58 | 59 |
| 2 | 300 | 0 | BEER | 38 | 39 | 41 | 44 | 46 | 47 | 49 | 52 | 53 | 56 | 57 | 60 |
| 3 | 0 | 35 | GIN | 3 | 4 | 8 | 10 | 15 | 17 | 20 | 22 | 27 | 30 | 32 | 36 |
| 4 | 300 | 35 | RUM | 2 | 5 | 9 | 12 | 13 | 18 | 21 | 24 | 25 | 29 | 31 | 35 |
| 5 | 150 | 35 | SAKE | 1 | 6 | 7 | 11 | 14 | 16 | 19 | 23 | 26 | 28 | 33 | 34 |

Each pen contained a Plasson water fountain and a 20.4 kg capacity feed tube. The dimensions of each pen were 5′×4′ with a stocking density of 622 cm² per bird when there were 30 broilers per pen.

The animals were started on new wood shavings. On day 5, all 60 pens received 226 g of used litter sourced from chickens not exposed to dietary DFM's or enzymes.

A total of 1800 (30/pen) 1-day-old Ross 308 AP straight run chicks were used.

On the first day of the study, 200 randomly caught chicks were weighed and an average weight determined. Then, the chicks were distributed to pens beginning with pen 1 (Table 2), 30 chicks were randomly taken from crates and group weighed so that all 60 groups of 30 chicks were within ±10% of the determined average weight. Lighting was provided to the chicks during the whole study. All pens were checked at least once daily during the study to control the availability of feed and water and the temperature.

Each feed type (Starter, Grower, Finisher) was prepared from a single large basal diet each formulated for each diet and treatment group to maintain equal sodium levels across each treatment group as much as possible.

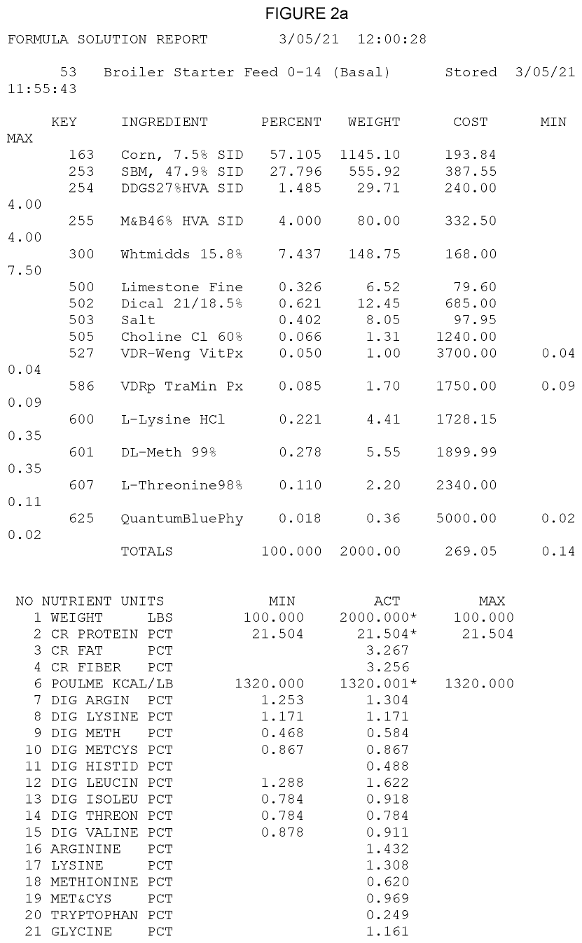

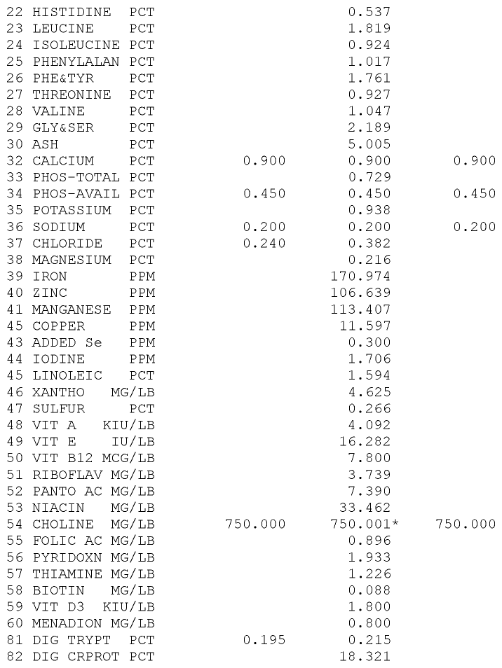

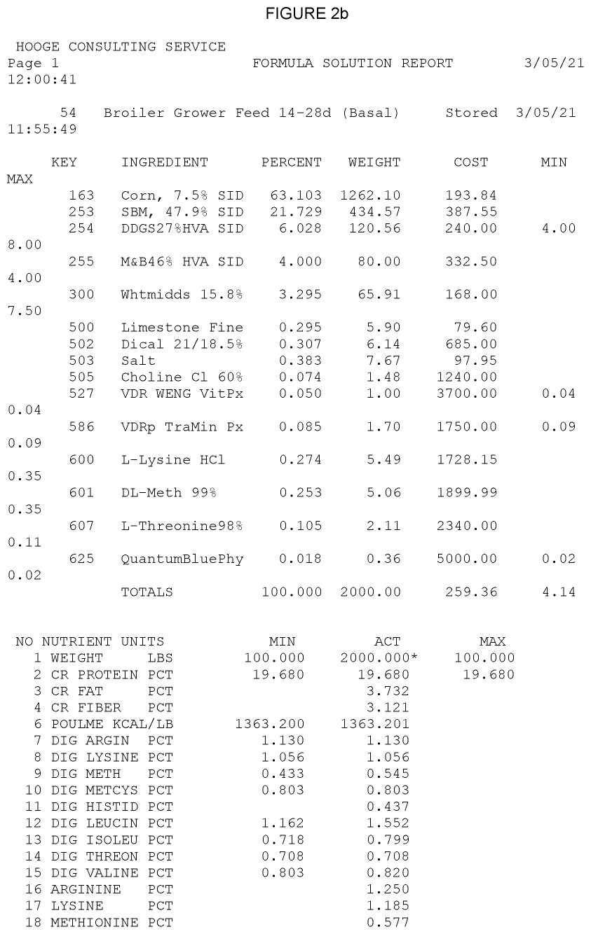

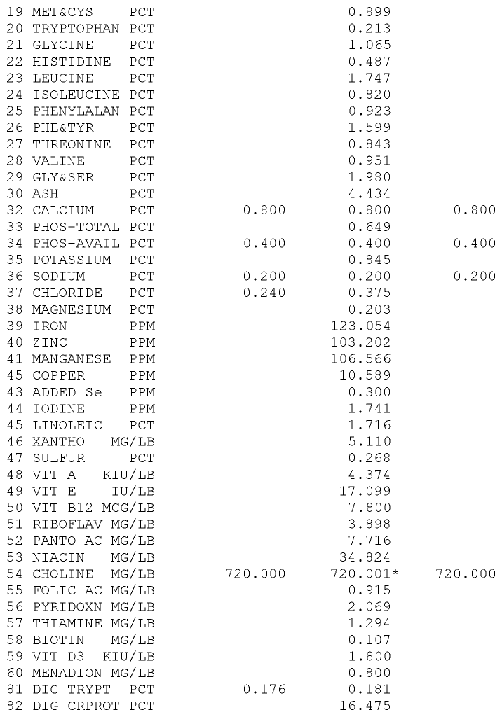

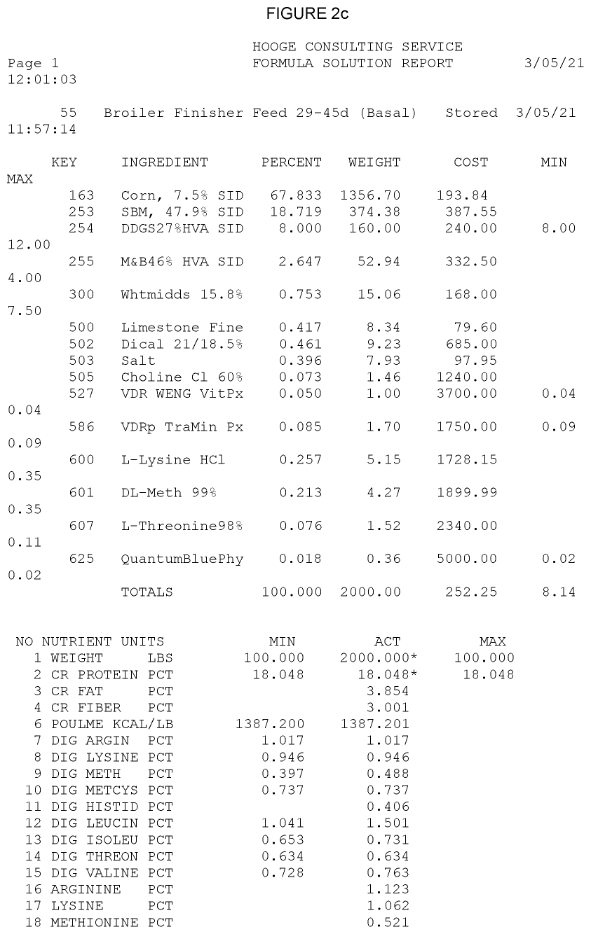

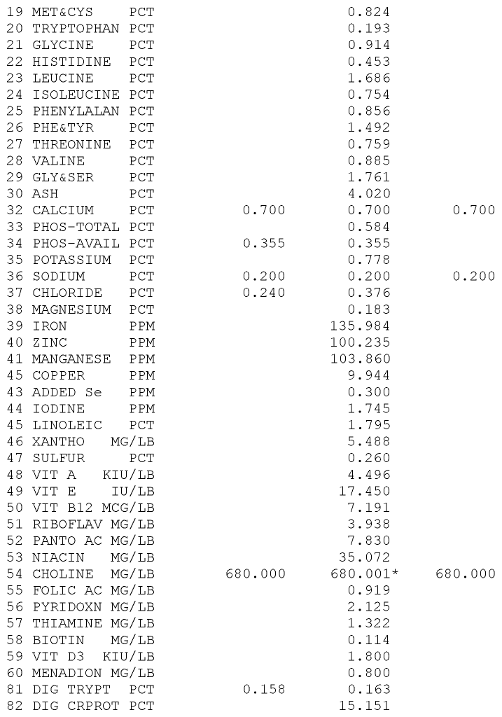

The diet was provided by age of bird (Starter from 0 to 14 days of age, Grower from 15 to 28 days of age and Finisher from 29 to 42 days of age). Feed formulas are reported in FIGS. 2a (Starter), 2b (Grower) and 2c (Finisher).

All feeds were mixed on site and the expected amount of each feed type to be allocated per bird was up to 567 g of Starter, up to 1590 g of Grower and up to 2270 g of Finisher.

The descriptions of the supplemented diets provided to the 5 study groups are reported in the following Table 3.

TABLE 3

| Group | Code | Starter (0-14 days) | Grower (15-28 days) | Finisher (29-42 days) |
|---|---|---|---|---|
| 1 | WINE | No supplements | No supplements | No supplements |
| 2 | BEER | 300 ppm ButiPEARL | 300 ppm ButiPEARL | 300 ppm ButiPEARL |
| 3 | GIN | 35 ppm ME10 | 35 ppm ME10 | 35 ppm ME10 |
| 4 | RUM | 300 ppm ButiPEARL + | 300 ppm ButiPEARL + | 300 ppm ButiPEARL + |
| | | 35 ppm ME10 | 35 ppm ME10 | 35 ppm ME10 |
| 5 | SAKE | 150 ppm ButiPEARL + | 150 ppm ButiPEARL + | 150 ppm ButiPEARL + |
| | | 35 ppm ME10 | 35 ppm ME10 | 35 ppm ME10 |

ButiPEARL—commercial formulation of encapsulated butyrate
ME10—commercial formulation of E. faecium NCIMB 10415

All broilers were weighed by pen when bird ages were 0, 14, 28 and 42 days and the feed was weighed back at 14, 28 and 42 days.

During the study, broiler body weights and feed intakes at days 0, 14, 28 and 42 as well as the related feed conversion value (FCV) were measured.

The obtained results are reported in the following tables.

TABLE 4
0-14 days

| | | Day 14 | |
|---|---|---|---|
| Group | Treatment | Weight | FCV |
| 1 | control | 0.897 | 1.130^{b} |
| 2 | 300 ppm ButiPEARL | 0.890 | 1.151^{ab} |
| 3 | 35 ppm ME10 | 0.880 | 1.186^{a} |
| 4 | 300 ppm ButiPEARL + 35 ppm ME10 | 0.882 | 1.187^{a} |
| 5 | 150 ppm ButiPEARL + 35 ppm ME10 | 0.878 | 1.184^{a} |

| | | |
|---|---|---|
| RCB P-value*: | 0.3645 | 0.0323 |

*P-value from RCB ANOVA.
FCV = Feed Conversion Value
Superscripts “^{a}” and “^{b}” = significantly different between them (p ≤ 0.05)

TABLE 5
0-21 days

| | | Day 21 | |
|---|---|---|---|
| Group | Treatment | Weight | FCV |
| 1 | control | 1.676 | 1.328 |
| 2 | 300 ppm ButiPEARL | 1.691 | 1.285 |
| 3 | 35 ppm ME10 | 1.679 | 1.308 |
| 4 | 300 ppm ButiPEARL + 35 ppm ME10 | 1.685 | 1.308 |
| 5 | 150 ppm ButiPEARL + 35 ppm ME10 | 1.688 | 1.301 |

| | | |
|---|---|---|
| RCB P-value*: | 0.9622 | 0.1758 |

*P-value from RCB ANOVA.
FCV = Feed Conversion Value
Superscripts “a” and “b” = significantly different between them (p ≤ 0.05)

TABLE 6
0-28 days

| | | Weight | |
|---|---|---|---|
| Group | Treatment | Day 28 | FCV |
| 1 | control | 2.796^{b} | 1.510^{a} |
| 2 | 300 ppm ButiPEARL | 2.935^{a} | 1.435^{b} |
| 3 | 35 ppm ME10 | 2.968^{a} | 1.452^{b} |
| 4 | 300 ppm ButiPEARL + 35 ppm ME10 | 2.961^{a} | 1.451^{b} |
| 5 | 150 ppm ButiPEARL + 35 ppm ME10 | 2.953^{a} | 1.451^{b} |

| | | |
|---|---|---|
| RCB P-value*: | 0.0007 | 0.0000 |

*P-value from RCB ANOVA.
FCV = Feed Conversion Value
Superscripts “^{a}” and “^{b}” = significantly different between them (p ≤ 0.05)

TABLE 7
0-42 days

| | | Weight | |
|---|---|---|---|
| Group | Treatment | Day 42 | FCV |
| 1 | control | 5.163^{b} | 1.567 |
| 2 | 300 ppm ButiPEARL | 5.380^{a} | 1.513 |
| 3 | 35 ppm ME10 | 5.517^{a} | 1.545 |
| 4 | 300 ppm ButiPEARL + 35 ppm ME10 | 5.405^{a} | 1.557 |
| 5 | 150 ppm ButiPEARL + 35 ppm ME10 | 5.437^{a} | 1.545 |

| | | |
|---|---|---|
| RCB P-value*: | 0.0001 | 0.1352 |

*P-value from RCB ANOVA.
FCV = Feed Conversion Value
Superscripts “^{a}” and “^{b}” = significantly different between them (p ≤ 0.05)

Starting from day 28 a significant difference between the treatment and the control group is observed. At the end of the study (42 days) the combinations of probiotic and butyrate resulted in an average weight increase numerically higher than the butyrate group.

1. A method comprising the step of administering Enterococcus faecium in combination with butyric acid or salts thereof as a feed supplement to livestock or pets to improve enterocyte health and consequently wellness of the livestock or pets.

2. The method according to claim 1, wherein the Enterococcus faecium is a strain with deposit number NCIMB 10415.

3. The method according to claim 1, wherein the butyric acid or salts thereof is sodium butyrate.

4. The method according to claim 1, wherein the Enterococcus faecium is in microencapsulated form.

5. The method according to claim 1, wherein the butyric acid or salts thereof is in incapsulated form.

6. The method according to claim 1, wherein the feed supplement is administered to livestock, and wherein the livestock is selected from the group consisting of fattening turkeys, chickens, laying hens, sows and piglets both suckling and weaned, fattening pigs, milk cows, and calves.

7. A feed supplement containing Enterococcus faecium in combination with butyric acid or salts thereof for use to improve the enterocyte health and consequently the wellness of livestock and pets.

8. The feed supplement according to claim 7, wherein the Enterococcus faecium is a strain with deposit number NCIMB 10415.

9. The feed supplement according to claim 7, wherein the butyric acid or salts thereof is sodium butyrate.

10. The feed supplement according to claim 7, wherein the Enterococcus faecium is in microencapsulated form.

11. The feed supplement according to claim 7, wherein the butyric acid or salts thereof is in incapsulated form.

12. The feed supplement according to claim 7, wherein the livestock are fattening turkeys, chickens, laying hens, sows and piglets both suckling and weaned, fattening pigs, milk cows, calves and the pets are dogs and cats.

13. The method according to claim 1, wherein the feed supplement is administered to dogs or cats.

14. The method according to claim 2, wherein the Enterococcus faecium is in microencapsulated form.

15. The method according to claim 2, wherein the butyric acid or salts thereof is in incapsulated form.

16. The method according to claim 2, wherein the feed supplement is administered to livestock, and wherein the livestock is selected from the group consisting of fattening turkeys, chickens, laying hens, sows and piglets both suckling and weaned, fattening pigs, milk cows, and calves.

17. The method according to claim 2, wherein the feed supplement is administered to dogs or cats.

18. The method according to claim 3, wherein the Enterococcus faecium is in microencapsulated form.

19. The method according to claim 3, wherein the butyric acid or salts thereof is in incapsulated form.

20. The method according to claim 3, wherein the feed supplement is administered to livestock or pets selected from the group consisting of fattening turkeys, chickens, laying hens, sows and piglets both suckling and weaned, fattening pigs, milk cows, calves, dogs, and cats.
